(19) United States
(12) Patent Application Publication
(10) Pub. No.: US 2013/0035661 A1
(43) Pub. Date: Feb. 7, 2013

(54) FLUID DELIVERY DEVICES AND METHODS OF USE FOR COLLAPSIBLE FLUID CONTAINERS

(71) Applicant: Adam David Tibble, Davis, CA (US)

(72) Inventor: Adam David Tibble, Davis, CA (US)

(21) Appl. No.: 13/565,575

(22) Filed: Aug. 2, 2012

Related U.S. Application Data
(60) Provisional application No. 61/574,452, filed on Aug. 2, 2011.

(51) Int. Cl. A61M 5/00

(52) U.S. Cl. 604/500; 604/131

(57) ABSTRACT

The present invention recognizes that there exits a need for a device and method to extrude fluid from a collapsible fluid bag in and uncomplicated manner using pressure, preferably to prevent or reduce the introduction of air into the fluid. A first aspect of the present invention is a medical device for administering fluid to a subject from a flexible fluid container. A second aspect of the present invention is a method of administering a fluid to a subject using a device of the present invention.

This application claims benefit of priority to U.S. Provisional Application, Ser. No. 61/574,452, filed Aug. 2, 2011, entitled “Fluid Delivery Devices and Methods of Use for Collapsible Fluid Containers,” which is incorporated by reference herein.

### TECHNICAL FIELD

The present invention generally relates to the field of situations in which it is desirable to extrude fluid from a collapsible fluid bag by using pressure, preferably to prevent or reduce the introduction of air into the fluid extruded from such collapsible fluid bag. Applicable situations include, but are not limited to, the medical field where medications, intravenous fluids, blood, blood products and other products are prepared within collapsible fluid vesicles. Although the description to follow primarily relates to the medical field, the invention applies to any number of situations where “hands free” pressure based fluid delivery from a collapsible entity is desirable. This includes, but is not limited to, the food industry, scientific laboratories, veterinary medicine, and mechanical engineering.

### BACKGROUND

Currently, fluid delivery in medicine is primarily accomplished by one of three mechanisms. All of these mechanisms and methods suffer from limitations that are addressed by the present invention.

First, gravity represents the most common mechanism of fluid administration. A vesicle is hung above the level of a patient's heart on a normal or specialized pole [Schmuhl J U.S. Pat. No. 5,135,191. Medical Support System. May 1991], while plastic tubing and an intravascular catheter provide a conduit for the fluid to “drip” into the patient. Although this method leaves a clinician's hands free to perform other tasks, the rate of fluid administration is limited by the height of the vesicle and the size of the intravascular catheter. The rate of flow rarely approaches what is necessary for adequate patient care in certain scenarios, such a trauma and surgical situations including blood loss. Moreover, should air become entrained within the fluid delivery circuit, the potential for life-threatening intravascular air embolism exists, placing the patient at risk for stroke or cardiovascular collapse [Adhikary G. Massive Air Embolism: A Case Report. Journal of Clinical Anesthesia. February 1998. Pgs 70-72].

Second, mechanical pumps exist that apply sequential pressure to specialized intravascular fluid delivery lines, the small plastic tubes running into the patient [O'Leary. U.S. Pat. No. 5,741,121. IV Fluid Delivery System. April 1998]. Although effective, these mechanical and electrical pumps and associated lines are cumbersome to “spike” and to set up for use (programming is required to rapidly increase fluid flow for a duration of time), are difficult to attach to a fluid warmer should the patient's condition require warmed fluids, are expensive, are prone to electrical and mechanical failure, and have a limited infusion rate, (The limited infusion rate is noted because an electrical pump cannot approach fluid rates achieved by the present invention which are important for certain clinical situations).

Third, pressure based solutions exist that entail placing the delivery fluid snugly next to a bladder [Bellin M U.S. Pat. No. 4,735,613. Pressure Infusion Device. April 1988]. The bladder is either electrically/pneumatically inflated (for example “Level 1 Transfusor” (Level 1 H-1200—Smiths Medical)) or mechanically inflated “by hand” (for example, IV pressure bag (Pressure Infusor—Baxter Medical)) to pressures approaching 300 mmHg. Inflation of the bladder impinges on the entire space occupied by the delivery fluid and forces fluid into the patient. Although this method provides fluid at an adequate rate, the set-up is often difficult, cumbersome, and time consuming. With certain types of fluid infusions, this method places the patient at significant risk for air embolism resulting in possible stroke or cardiovascular collapse, especially in the setting of inadequate “de-airing” of the delivery fluid bag [(Pant D. Significant Air Embolism: A possibility even with collapsible intravenous fluid containers when used with a rapid infuser system. Indian Journal of Anesthesia January-February 2010. Pgs 49-51) AND (Linden J. Fatal Air Embolism Due to Perioperative Blood Recovery. Anesthesia & Analgesia. Vol 84; 1997. Pgs 422-6)].

Drawing from the strengths and weakness of the current mechanisms outlined above, a preferred fluid delivery mechanism can have the characteristics of: 1) sets up easily in an uncomplicated fashion; 2) delivers fluid quickly (an exact ml/min rate may not be as important to the clinician as an “eyeball” test on the infusion line to see that the fluid is “dripping” quickly); 3) frees the clinician's hands for performing other important patient care duties; and 4) provides some amount of protection against air embolism beyond the meticulous “de-airing” of the fluid delivery bag. In contrast to the state of the art, as exemplified by [(Schaffer I. U.S. Pat. No. 4,684,367. Ambulatory Intravenous Delivery System. August 1987) AND (Peterson D. patent application Ser. No. 10/677,718. Apparatus and Method of Intravenous Fluid Infusion. April 2004) AND (Yoshioka W U.S. Pat. No. 6,558,346. Automatic Control-Type, Portable Intravenous Infusion Apparatus and Jacket Therefore. May 2003) AND (Kleeman M U.S. Pat. No. 6,669,668. Medication Delivery Pump. December 2003) AND (West J. U.S. Pat. No. 6,062,429. Apparatus for Supporting and Discharging Flexible Fluid Containers. May 2000) AND (Bellin M U.S. Pat. No. 4,735,613. Pressure Infusion Device. April 1988)], the present invention addresses these four characteristics in a simple, elegant, cost-effective and unique fashion, and provides related benefits as well.

### BRIEF DESCRIPTION OF THE FIGURES

FIG. 1 depicts one aspect of the present invention where two faceplates are connected by a hinge applying pressure to a fluid delivery bag with the aid of spring clamps. Certain non-limiting aspects of FIG. 1 are described in Example 1.

FIG. 2 depicts another aspect of the present invention where two faceplates are connected by a dual hinge element that can span the length of the faceplates. Certain non-limiting aspects FIG. 2 are described in Example 2.

FIG. 3A and FIG. 3B depict a further aspect of the present invention where a constant tension torsion spring replaces the spring clamps in prior figures. Certain non-limiting aspects of FIG. 3A and FIG. 3B are described in Example 3.

FIG. 4A and FIG. 4B depicts yet another aspect of the present invention where a constant tension torsion spring is attached to support elements that faceplates engage, or “snap” into. Certain non-limiting aspects of FIG. 4A and FIG. 4B are described in Example 4.

FIG. 5 depicts another aspect of the present invention where handles are used to facilitate faceplate separation. Certain non-limiting aspects of FIG. 5 are described in Example 6.

FIG. 6 depicts a further aspect of the present invention where the article of manufacture of the present invention is used in a “bottom-up” configuration. Certain non-limiting aspects of FIG. 6 are described in Example 11.

FIG. 7A and FIG. 7B depict yet another aspect of the present invention where the article of manufacture of the present invention is provided in a “top to bottom” pressurization configuration with one valve configuration. Certain non-limiting aspects of FIG. 7A and FIG. 7B are described in Example 17.

FIG. 8A, FIG. 8B and FIG. 8C depict a further aspect of the present invention, particularly the Pin & Cable locking mechanism in front and back views. Certain non-limiting aspects of FIG. 8A, FIG. 8B and FIG. 8C are described in Example 19.

FIG. 9A, FIG. 9B, FIG. 9C, FIG. 9D, FIG. 9E and FIG. 9F depict another aspect of the present invention, particularly the Rod & Channel locking mechanism in various views and operational configurations. Certain non-limiting aspects of FIG. 9A, FIG. 9B, FIG. 9C, FIG. 9D and FIG. 9E are described in Example 20. In particular, FIG. 9A and FIG. 9B depicts the structure in a closed configuration with a channel in different views. FIG. 9C depicts the structure in an open configuration. FIG. 9D depicts another view of the structure in an open configuration. FIG. 9E depicts a close up of a notch structure provided in the article. FIG. 9F depicts an article of manufacture of the present invention, inclusive of face plates.

### SUMMARY

The present invention recognizes that there exits a need for a device and method to extrude fluid from a collapsible fluid bag in an uncomplicated manner using pressure, preferably to prevent or reduce the introduction of air into the fluid.

A first aspect of the present invention is a medical device for administering fluid to a subject from a flexible fluid container

A second aspect of the present invention is a method of administering a fluid to a subject using a device of the present invention.

### DETAILED DESCRIPTION OF THE INVENTION

### Definitions

Unless defined otherwise, all technical and scientific terms used herein have the same meaning as commonly understood by one of ordinary skill in the art to which this invention belongs. Generally, the nomenclature used herein and the procedures referred to in medicine described below are well known and commonly employed in the art. Conventional methods are used for these procedures, such as those provided in the art and various general references such as [Miller R. Miller's Anesthesia. 6^{th} Edition. Elsevier. 2005]. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

Other technical terms used herein have their ordinary meaning in the art that they are used, as exemplified by a variety of technical dictionaries.

### Introduction

The present invention recognizes that there exits a need for a device and method to extrude fluid from a collapsible fluid bag in an uncomplicated manner using pressure, preferably to prevent or reduce the introduction of air into the fluid.

As a non-limiting introduction to the breath of the present invention, the present invention includes several general and useful aspects, including:
1) A medical device for administering a fluid to a subject from a flexible fluid container; and
2) A method of administering a fluid to a subject using a medical device of the present invention.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

I A Medical Device for Administering a Fluid to a Subject from a Flexible Fluid Container

The present invention includes a medical device for administering at least one fluid to at least one subject from at least one flexible fluid container, including: a) at least two plates, each having a front face, a back face, a top edge, a bottom edge, a right edge, and a left edge; wherein the top edges of the at least two plates are operably engaged with one or more hinge means; and b) at least one pressure inducing means; wherein said pressure inducing means is operably engaged with said at least two plates to provide pressure to said at least two plates.

The fluid can be any fluid that is to be administered to a subject in need of such fluid. Examples include, but are not limited to blood, serum, fluids routinely provided to a subject for a variety of purposes, such as lactated Ringers, or a combination thereof, The fluid can be supplemented with additional agents as desired by the user of the medical device of the present invention, such as but not limited to, antibiotics, chemotherapeutic agents, antibodies, drugs, or other agents.

The subject can be any subject in need of such fluid. The subject can be a human or non-human primate, a veterinary animal, any companion animal, and the like. In general, the subject can be any living organism, preferably a higher organism, more preferably a vertebrate, but that need not be the case.

The flexible fluid container is preferably one for use to administer a fluid to a subject and is of appropriate size and shape for the type, amount and size the fluid container, as well as the character of the subject. For human and veterinary applications in particular, especially human applications, there are a variety of commercially available flexible fluid containers. Baxter, for example, produces and commercially sells such flexible fluid containers, but other manufacturers do so as well. In a preferred aspect of the present invention, the flexible fluid container is taken off the shelf and used with the present invention, but that need not be the case

The plates and other elements of the present invention can be made of any appropriate material, and can be the same or different, either separately or within an element. Appropriate materials include, but are not limited to plastic, one or more polymers or copolymers, glass, ceramics, metal, a combination thereof, and the like. The choice of material is dependent upon, for example, the environment of intended use. For example, glass may not be particularly suited for trauma unit applications where broken glass is a hazard, as are sharp edges. Also, the physical characteristics of the material can be considered, such as force applied, where some materials may become damaged at certain pressures. Also, for some applications, the material is preferably of medical grade. Transparency or relative transparency of the material may also be a factor to consider, as the user may wish to be able to view through at least one of the one or more plates, but this need not be the ease.

In one aspect of the present invention, when at least one flexible fluid container is provided between the at least two plates, pressure is applied to the at least two plates by pressure inducing means to the flexible fluid container to expel fluid from an outlet of said flexible fluid container. The amount and duration of pressure, or variable pressure, or no or substantially no pressure applied over time, is dependent upon the environment that the medical device of the present invention is used. For example, under trauma conditions where a subject has experienced significant fluid loss, such as blood, the amount of pressure may be relatively high. Under conditions of routine surgery where little fluid loss is expected or experience, the amount and duration of the amount of pressure applied may be relatively low, and in some cases may be stopped for a period of time dependent upon the condition of the subject.

In another aspect of the present invention, the one or more hinge means are the same or different and is selected from the group of metal, tape, ties, plastic ties, springs or rings, and can be the same or different or a combination thereof. The choice of hinge means depends on the environment of use of the medical device of the present invention. For example, metal hinges may not be appropriate under some surgical conditions as not being of medical grade material and have potentially sharp edges. In other applications, ties may not have the strength to withstand the pressure under which the medical device of the present invention is operated.

In yet another aspect of the present invention the pressure inducing means is selected from the group consisting of one or more clamps, one or more springs, one or more magnets, one or more rubber bands, and one or more bungee cords, and can be the same or different or a combination thereof. The choice of pressure inducing means depends on the environment of use of the medical device of the present invention. For example, metal clamps may not be appropriate under some surgical conditions as not being of medical grade material and have potentially sharp edges. In other applications, rubber bands may not have the strength to provide or withstand the pressure under which the medical device of the present invention is operated.

In another aspect of the present invention gas from the flexible fluid container does not substantially exit the flexible fluid container. In this aspect of the present invention, the prevention of gas exiting the flexible fluid container can be important as introduction of gas into the subject may cause unfortunate medical consequences, such as the damage of tissues or organs of the subject

In an additional aspect of the present invention, the one or more hinge means is or are placed in a position such that it does not substantially interfere with an existing hanging apparatus of a fluid delivery bag. This aspect of the invention is important under certain conditions of use, as it can in some instances be undesirable for the hinge means to interfere with the operation of a hanging apparatus of a fluid delivery bag, such as commercially available poles and the like for use in medical situations, but this need not be the case. In such situations, the user of the medical device of the present invention may find it cumbersome to utilize the medical device of the present invention. This can be important under situations where time is of the essence, but that need not be the case.

Furthermore, the one or more hinge means can be placed anywhere along the plates, including centrally located, located off centered, partially along the plates, or completely or substantially completely along the plates. The choice of location of the hinge means is also dependent upon the environment of intended use of the article of manufacture of the present invention. For example, a hinge means that completely or substantially completely passes along the plates may produce more force or be more reliable as opposed to configurations where that is not true, but that need not be the case.

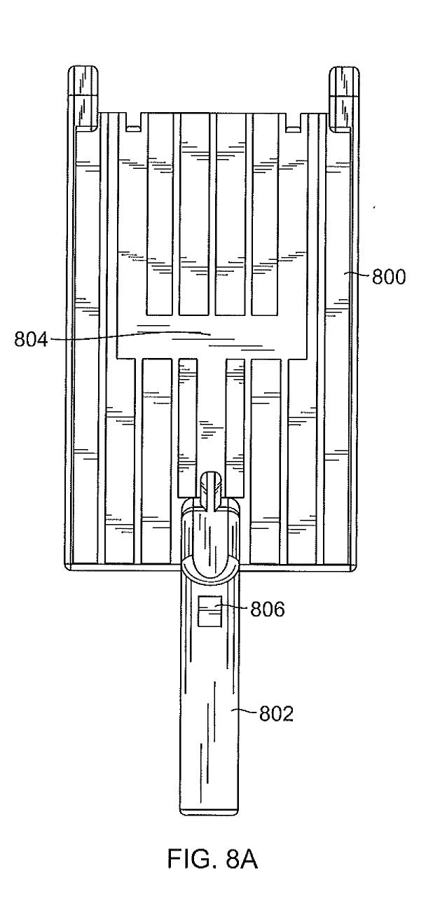

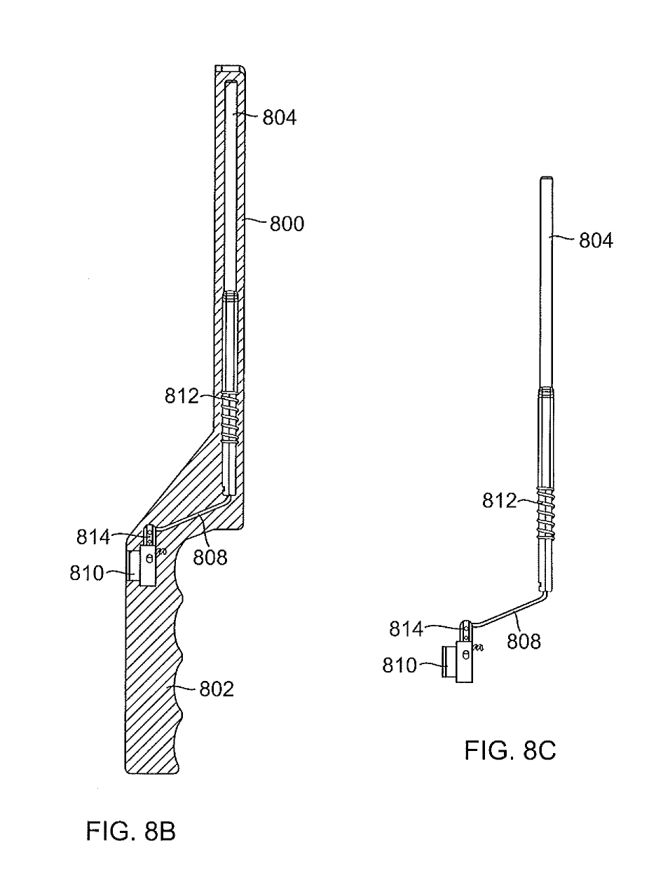

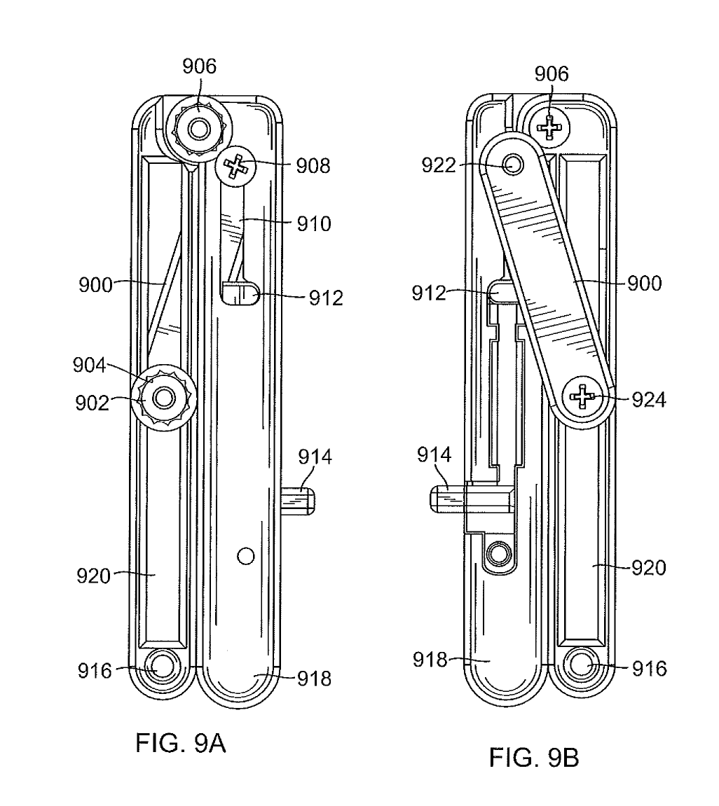

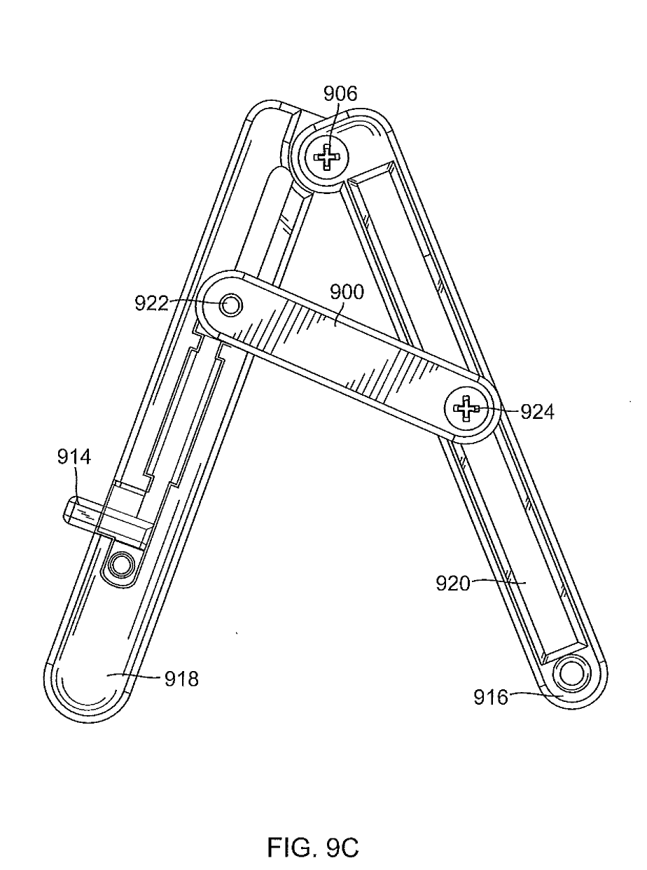

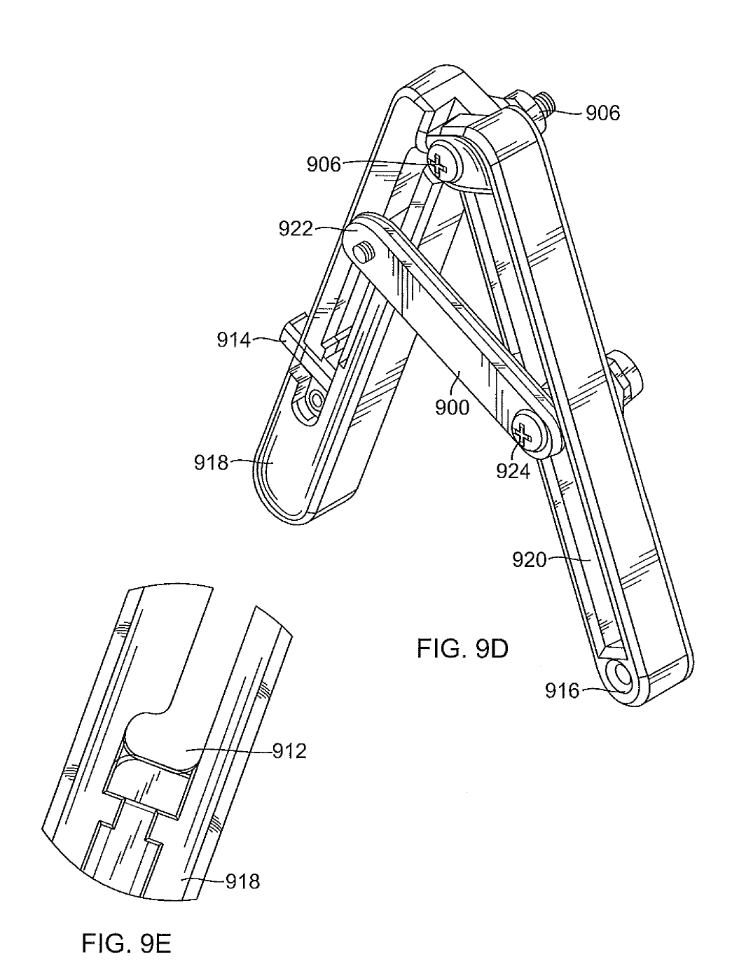

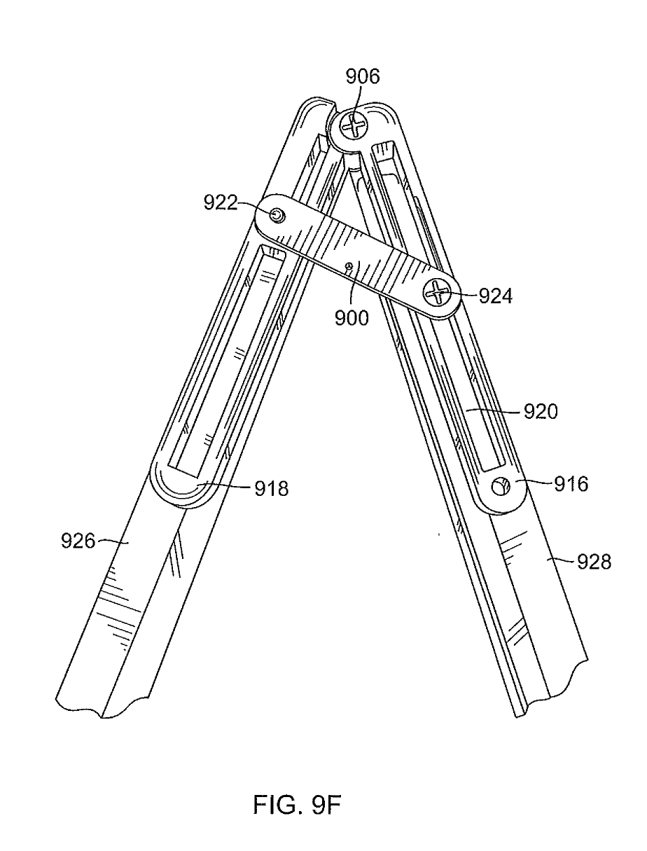

In yet another aspect of the present invention, the medical device of the present invention includes at least one trigger mechanism, the use of which can the safety and efficiency of said medical device. The trigger mechanism can be a pin and cable locking mechanism, a rod and channel locking mechanism, or a combination of both. Various aspects of such trigger mechanisms are described in the Figures and the Examples. For example, a pin and cable locking mechanism of the present invention is described in Example 19, along with FIG. 8A, FIG. 8B and FIG. 8C, as well as elsewhere in the present document. Likewise, a rod and channel locking mechanism of the present invention is described in Example 20, along with FIG. 9A, FIG. 9B, FIG. 9C, FIG. 9D, FIG. 9E and FIG. 9F. However, the invention is not intended to be limited by that Example, Figures or particular passages of the present document. The trigger mechanism can increase the safety and efficiency of the medical device of the present invention by making the operation of the device of the present invention operate more predictably and efficiently by providing, for example, increased reliability, such as, but not limited to, pressure application to the flexible fluid container, thus providing more predicable fluid flow there from, but that need not be the case.

### II A Method of Administering a Fluid to a Subject Using a Medical Device of the Present Invention

The present invention also includes a method of administering at least one fluid to at least one subject, including: a) providing at least one subject in need of said at least one fluid; b) providing at least one medical device of the present invention; c) providing at least one flexible fluid container comprising at least one fluid to be administered to the at least one subject; and d) operably engaging said at least one flexible fluid container with the at least one medical device of the present invention and the at least one subject to allow the flow of the at least one fluid to the at least one subject from the at least one flexible fluid container.

Any appropriate structure can connect the flexible fluid container to the subject, preferably a tube and needle made of medical grade materials, but that need not be the case. If the subject is to be administered the fluid orally, a needle need not be present. There can be one or more of such appropriate structures

Any appropriate materials for any element of the present invention as described herein can be utilized. In a preferred aspect of the present invention, a flexible fluid container known in the art is provided along with a medical device of the present invention, along with associated materials for administering fluid to the subject, such as tubes and in some instances needles, and the fluid is administered to the subject by the art recognized methods, such as but not limited to, by intravenous administration, interparitoneal administration, oral administration, rectal administration, direct administration to a tissue or organ, or other routs of administration as desired.

### EXAMPLES

### Example 1

### Basic Concept and Construction

For basic construction of the device one places a hinge element connecting two faceplates. The hinge element may consist of any appropriate hinge means, such as, but not limited to a standard hinge that one may find in a door frame, or any other connection method, such as tape, metal rings, plastic ties, or rope ties. The article of manufacture of the present invention may include a hinge in an off-center position (see, this Example 1) or use a central or dual hinge design (see, Example 2). In the off-center model, one sets the hinge element in a position such that it does not encroach upon the existing hanging apparatus of the fluid delivery bag. Since the fluid delivery bag is often hanging on a separate pole by a central hole, this usually necessitates placement of the hinge element in an off center position. The off-center hinge allows the device to jacket around the already hanging fluid bag. In contrast to other pressurizing fluid delivery options which require removal of the fluid bag and cumbersome placement within a chamber near the pressurization bladder [Benin M. U.S. Pat. No. 4,735,613. Pressure Infusion Device. April 1988], the present invention avoids this step by allowing placement upon an already hanging fluid bag. Therefore placement of the device of the present invention is more efficient and less cumbersome than those known and used in the art.

After the two flat faceplates are connected by a hinge element, one jackets the device around a fluid bag, which may be already hanging at an appropriate height. Then one places two constant tension spring clamps, such as industrial strength clamps readily available on the market, such as hardware stores, on the device, one at the hinge, and another equally spaced between the hinge side and opposite side. One chooses specific constant tension spring clamps such that they provide significant pressure for compressing the faceplates towards one another at an adequate rate, thereby forcing fluid from the delivery bag and into the patient for the desired clinical effect, More specifically the clamp springs utilized are preferably torsion springs, those that store mechanical energy when twisted. These types of springs are found commonly in industrial strength clamps that are readily available at most hardware stores. Another example of torsion springs would be their use in the everyday mousetrap, where mechanical energy is stored upon twisting of the spring; and their kinetic energy is released by trap triggering. Similarly, torsion springs utilized in the present invention would gradually leak their stored mechanical energy into kinetic energy as the associated opposing faceplates collapse upon one another.

The choice and construction of the faceplate is within the skill of the ordinary artisan. One chooses material rigid enough to transmit the pressure provided by the industrial strength clamps across the entire area of the faceplate. Additionally, one can reinforce the faceplate with material to achieve an appropriate stiffness for transmission of this pressure throughout the faceplate, and thus throughout the entire side of the delivery fluid bag that is in contact with the faceplate. Preferable materials for use include, but are not limited to, plexiglass, glass, metal, and medical grade plastic (non PVC—for example, medical grade polypropylene).

Moreover, the size of faceplates one uses represents one aspect of the invention. In the medical field, delivery fluid bags invariably come in standardized sizes and volumes. For example, packed red blood cells (pRBCs) and fresh frozen plasma (FFP) come in a standardized 500 ml fluid bag. For design and construction of a device of the present invention, one uses faceplates sized appropriately for the desired delivery fluid. For example, the faceplate used for pRBCs can be sized slightly shorter than the length of the 500 ml pRBC bag. The shortened length of the faceplate is preferable in that pressure will not be transmitted to the fluid delivery bag for this short distance. If the fluid delivery bag contains a small amount of air, the shortened faceplate will provide a certain safety volume of appropriate volume, such as a few milliliters, that will not be pressurized into the patient, thus protecting against the life-threatening phenomenon of air embolism.

The present invention also provides options for the color and transparency of the faceplates ranging from clear to color-coding for specific sized fluid vesicles. In addition, the faceplates or other portions of the device of the present invention can be provided with markings, indicia, formulas, or writing space for the convenience of the user.

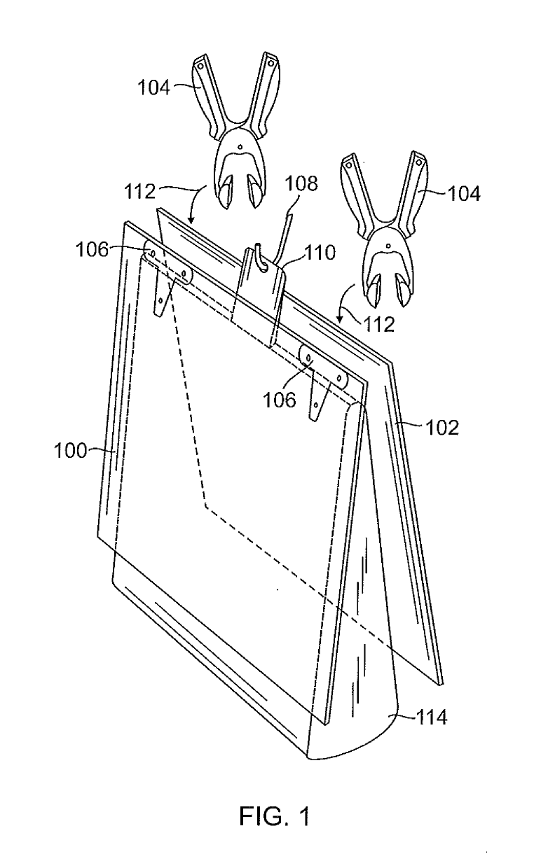

FIG. 1 depicts preferred aspects of the present example and invention is provided herewith. The figure is intended for illustrative purposes and is not intended to limit the present invention. Depicted are a first plate (100) and a second face plate (102) operably engaged with two hinges (106) with clamps as pressure means (104) with arrows (112) depicting how and where the clamps can engage the structure. Also depicted is a hanging element (110) to engage a hook (108), with a flexible fluid container (114) also depicted.

### Example 2

The basic concept described in Example 1 is modified to include two faceplates connected by a dual hinge element that spans the length of the faceplates.

In this aspect of the present invention a dual hinge or long central hinge replaces the off-center hinge described in Example 1. Although the device would not preferably be jacketed around an already hanging fluid bag, the present invention can be hung from an existing IV pole (Frinzel J. U.S. Pat. No. 4,113,222. Intravenous Pole. September 1978) and contain a recessed hook on the inside of the pressure plates in order to hang the fluid bag. Although the user must place the fluid bag within the device, in contrast to Example 1 where it jackets around an already hanging fluid bag, the dual hinge or central hinge design still avoids the cumbersome process of stringing a nylon strap through the fluid bag hole and pneumatic bladder inflation by hand pumping as is the current procedure required with the prior art (Pressure Infusor—Baxter Medical).

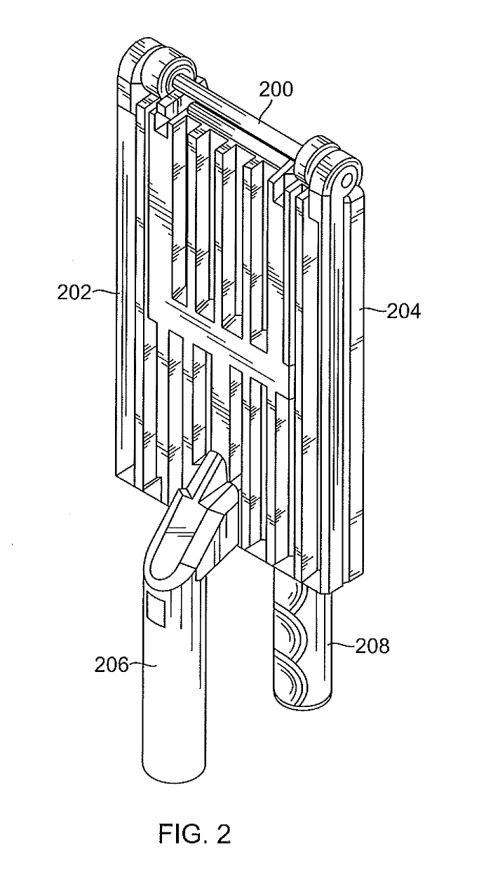

FIG. 2 depicts preferred aspects of the present example and invention is provided herewith. The figure is intended for illustrative purposes and is not intended to limit the present invention. Depicted are a dual hinge or long central hinge element (200) operably engaging a first plate (202) and a second plate (204). Also shown are a first extended handle (206) and a second extended handle (208) engaging the first plate (202) and the second plate (204).

### Example 3

### Replacement of Industrial Strength Clamps with a Constant Tension Spring

In this aspect of the present invention, one places a constant tension spring above the hinge element or in place of the hinge element in off-center manner (see, Example 1) or centrally (see, this Example 2). The constant tension spring is attached to the opposing faceplates such that pressure is transmitted equally to each faceplate, urging them to close upon one another. The spring tension is chosen such that fluid is delivered at the desired speed for optimal patient care.

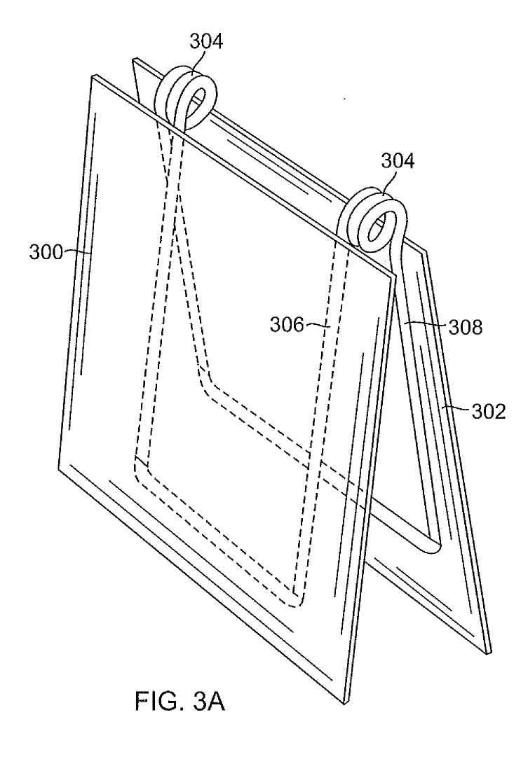

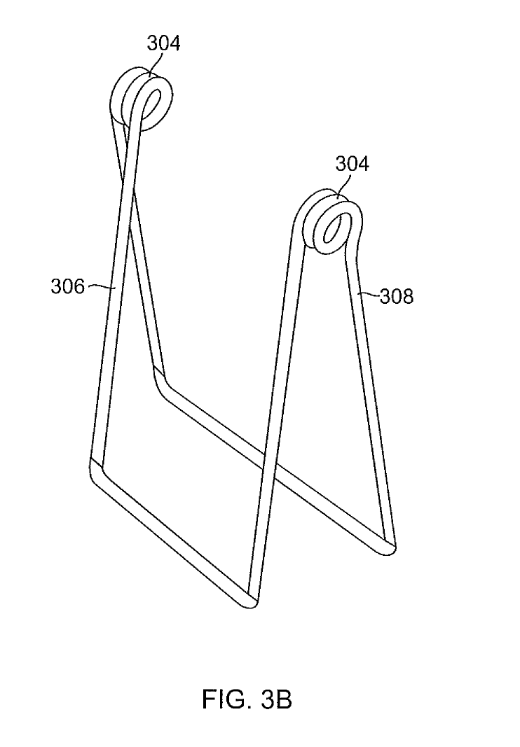

FIG. 3A and FIG. 3B depict preferred aspects of the present example and invention is provided herewith. The figure is intended for illustrative purposes and is not intended to limit the present invention. Depicted in FIG. 3A are a first plate (300) and a second plate (302) engaged with a dual spring structure (304) with a first plate engaging structure (306) and a second plate engaging structure (308). FIG. 3B depicts the dual spring structure (304) with the first plate engaging structure (306) and second plate engaging structure (308) in the absence of plates (300 and 302 in FIG. 3A).

### Example 4

### Tension Spring Attached to Support Elements that Faceplates can “Snap” into

In this aspect of the present invention, one attaches the tension spring to appropriately sized support elements. Concurrently, faceplates are constructed in such a fashion that allows temporary attachment to the support elements of the constant tension spring. This allows for faceplates to be removed and replaced should it become necessary for patient care, such as, for example, cleanliness, sanitation, a new patient, and the like.

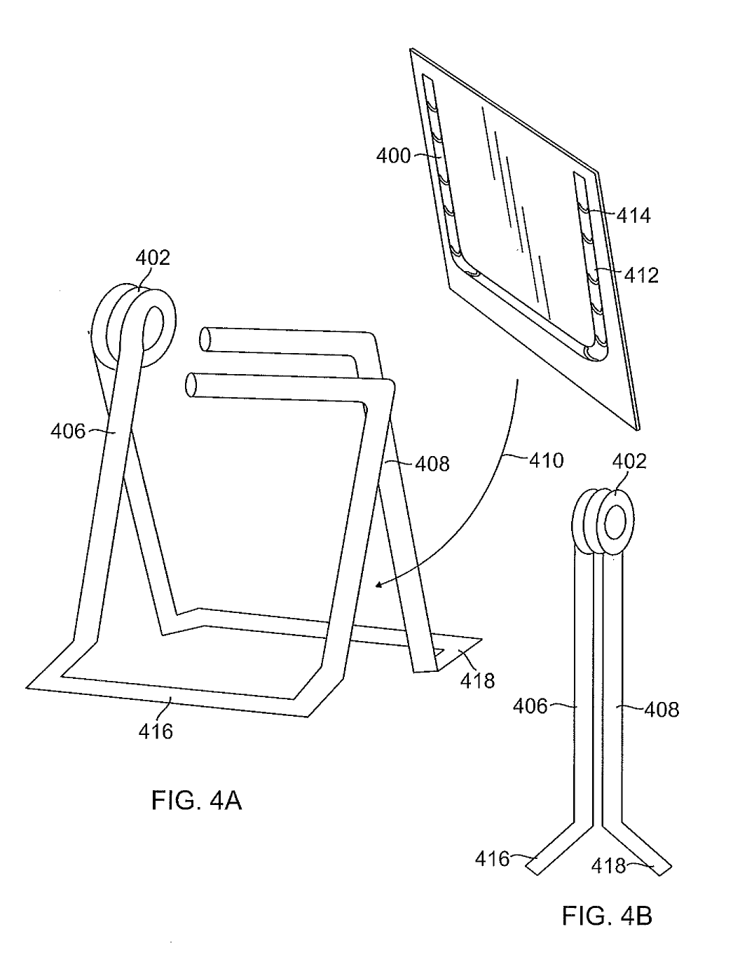

FIG. 4A and FIG. 4B depict preferred aspects of the present example and invention is provided herewith. The figure is intended for illustrative purposes and is not intended to limit the present invention. FIG. 4A depicts a plate (400) with a grove (412) with ridges (414) that allow the plate to snap into (depicted by arrow 410) a single spring plate engaging structure having a single spring (402) and a first plate engaging structure (406) and a second plate engaging structure (408) that are non-contiguous and have a first flange (416) and a second flange (418). In operation, there would be two plates. FIG. 4B depicts a side view of the single spring plate engaging structure of FIG. 4A in a perspective view. The single spring (402), first plate engaging structure (406) and second engaging structure (408) along with the first flange (416) and second flange (418). Plates are not shown in this view.

### Example 5

### Support Elements Attached to Tension Spring are Constructed with a Slide Element—Allowing them to Accommodate Various Width/Length of Chosen Faceplates

In order to accommodate various sized faceplates, one constructs the tension spring support element with a sliding mechanism—allowing them to “snap” into different sized faceplates, This sliding mechanism can increase or decrease the length or width of the faceplate support elements, thus allowing for a universal tension spring to attach to a multitude of faceplate sizes and styles.

### Example 6

### Addition of an Accessory that Aides in Separation of Faceplates

In order to place the apparatus onto an already hanging fluid bag, and for removal of the apparatus after completion of fluid infusion, an accessory is constructed such that it eases separation of faceplates given that they are under constant apposition pressure. The accessory can be in the form of long handles as an additional length attachment to the faceplates such that an increased moment arm eases faceplate separation. Or one utilizes a separate accessory with its own spring mechanism that attaches to the off center tension spring. Using opposing force, this accessory spring element opens the faceplates.

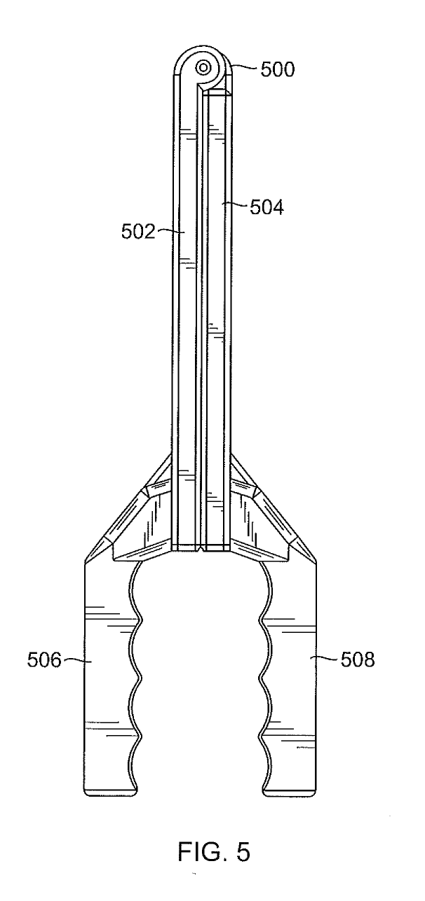

FIG. 5 depicts preferred aspects of the present example and invention is provided herewith. The figure is intended for illustrative purposes and is not intended to limit the present invention. This figure is essentially a side view of the device depicted in FIG, 2. Depicted are the hinge (500) with a first plate (502) and a second plate (504). A first long handle (506) and a second long handle (508) are also depicted.

### Example 7

### Faceplates with a “Flared” Edge

In this variation of the present invention, one constructs faceplates that have a flared distal edge (the edge opposite the off center tension spring). This flared distal edge not only provides a purchase for manual separation of the faceplates that are under constant opposing pressure, but also provides for a short distance of the fluid bag that does not see faceplate application pressure. Therefore, similar to a shortened faceplate in example 1, the flared faceplate provides a safety volume of a few milliliters that will not be pressurized into the patient, thus protecting against air embolism.

### Example 8

### Replacement of Constant Tension Spring with a Variable Tension Spring

In this form of the present invention, a variable tension spring allows the user to “dial in” or select a desired pressure or approximate or relative pressure that urges the two faceplates together. For example, a variable tension spring is chosen such that it can provide 100, 150, 200, 250, or 300 mmHg pressure to the fluid bag. The user may then choose a desired pressure by adjusting the variable tension spring.

### Example 9

### Curvilinear Faceplates Utilized Instead of Flat Faceplates

The present invention can utilize curvilinear faceplates in place of flat faceplates. One designs such curved faceplates so that a slight curve can result in desired pressure variations as seen by the fluid delivery bag as the curved faceplates close. This design can also include the flared edge to protect against air embolism as stated in Example 7.

### Example 10

### Faceplate Spacer Utilized to Protect Against Complete Closure, and Thus Protecting Against Air Embolism

In this form of the present invention, one includes a faceplate spacer that prevents complete apposition of the two faceplates. Most important to a clinician is that the majority of fluid within the fluid delivery bag makes its way into the patient in a timely fashion. A faceplate spacer prevents only a small amount of fluid (or air) from being extruded from the fluid delivery bag. The clinician's goals are realized since a majority of the fluid is still infused, and the patient is protected against air embolism by a small faceplate spacer. The spacers can be located at the bottom, top, or sides of the faceplates, for example.

### Example 11

### Inversion of the Apparatus to Compress the Fluid Delivery Bag from Top-Down, to Bottom-Up Configuration

The present invention and all variations can be used in an inverted fashion to compress a fluid delivery bag from bottom to top instead of top to bottom. Although convention may seem to dictate fluid infusion from top down, the invention described can be used to compress a fluid delivery bag from bottom to top. Fluid will still find the path of least resistance through the open tubing conduit “down” into the patient, while air can be pushed up towards the top of the bag secondary to its decreased density. This aspect of the invention provides additional protection against life threatening air embolism, especially if used in concert with previously stated modifications such as shortened faceplates, flared edges, or curvilinear faceplates.

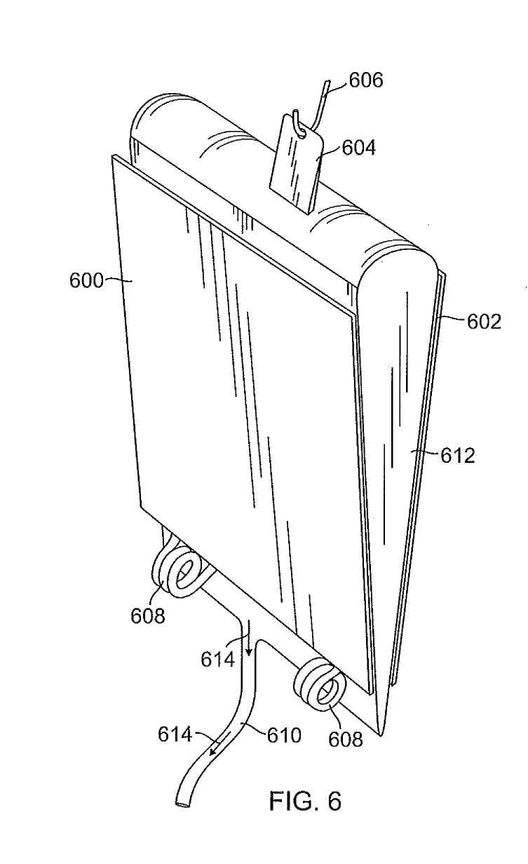

FIG. 6 depicts preferred aspects of the present example and invention is provided herewith. The figure is intended for illustrative purposes and is not intended to limit the present invention. Depicted are a first plate (600) and second plate (602) engaged with a flexible fluid container (612). The plates are engaged with a dual spring structure, springs shown as (608). Also depicted is a tube for fluid exit (610) from the flexible fluid container (612) with direction of flow show by arrows (614). Further depicted are a structure to engage a hook (604) and a hook (606).

### Example 12

### Addition of Pressure Sensor

The present invention can also optionally include a pressure sensor to quantify the force with which the faceplates tend to draw together. This may be in the form of a standard mmHg measure, or a color coded pressure sensor indicating adequate pressure or undesirable pressure levels (for example, too much or too little pressure).

### Example 13

### Formulation of a Closed Loop Feedback Mechanism for Optimal Pressure Generation

In this form, the invention can include a closed loop feedback mechanism that can increase or decrease faceplate apposition tension based on measurements of the present tension within the circuit. One can use this closed loop feedback mechanism in a situation where the apparatus tuned itself to a goal pressure, or to one that is “dialed in” by the user.

### Example 14

### Faceplate Modifications to Include Valuable Patient Care Information Displayed on the Faceplate

One can provide the faceplates to include information such as, but not limited to, scientific formulas, conversion charts, fluid formulas, allowable blood loss formulas, physiologic formulas, normal values charts for common labs, common intravenous fluid compositions, and the like. One may also include an interactive writable area or even an electronic tabulator that allows practitioners to circle or chart the patient's blood product utilization or fluid requirements.

### Example 15

### Use of Different Types of Force to Coax Faceplate Closure

The present invention can also utilize alternate methods or means of application of force (as opposed to springs or clamps) for faceplate closure. Magnetic force, for example could also draw faceplates together. Other possibilities include bungee cords and rubber bands.

### Example 16

### Placement of Apparatus from the Side (as Opposed to Top-Down or Bottom-Up Configurations)

On can place the apparatus described from the side, making placement of the invention easier and less cumbersome for the practitioner. Since the pressure applied to the collapsible fluid bag is dictated by the shape and design of the faceplates, one may place the hinge element of the invention “from the side,” so as to collapse the fluid bag from right-to-left or left-to-right. Regardless of the direction of fluid delivery bag compression (whether it be “top-down,” “bottom-up,” or “from the side”), the fluid will travel out of the only open conduit attached to the bag—the “spiked” or “nipple” end, which hangs from the dependent, or bottom, side of the fluid delivery bag. Therefore, placement of the pressure producing apparatus from the side is an option since it may ease use for the practitioner. Although there is a very small chance of air entrainment (as opposed to a “bottom-up” configuration), placement of the apparatus “from the side” is an option to provide the benefits of unhindered use outweighs the extremely small risk of air entrainment.

### Example 17

### Placement of a Valve on the Faceplates so as to Halt Fluid Flow at a Predetermined Distance from the End of the Fluid Delivery Bag

In this form of the present invention, a valve is fashioned on the faceplates such that the valve “pinches” off the fluid delivery bag at a predetermined distance from the end of the fluid delivery bag. Therefore, fluid flow ceases at the valve, despite pressure still being applied proximally. One places the faceplates and apparatus such that the valve prevents the final few milliliters of fluid from running into the patient, thus protecting against air embolism. Depending on the direction of application of the invention to the fluid bag (from the top, bottom, or side), this valve can be fashioned on the top, bottom, or side of the faceplate apparatus such that it “pinches” off the final few milliliters of fluid or air, protecting against entraining intravascular air.

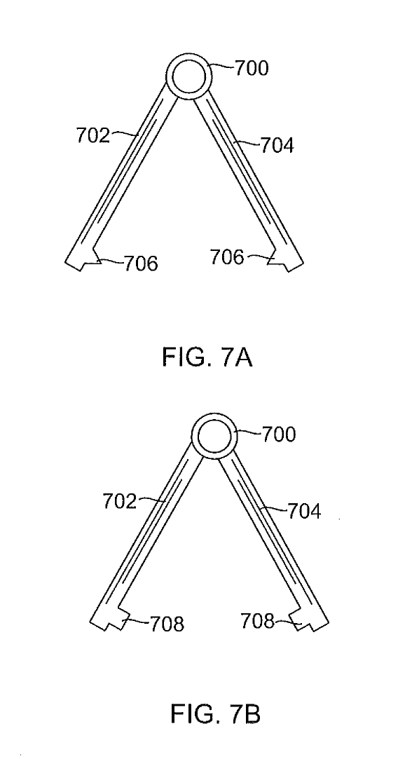

FIG. 7A and FIG. 7B depict preferred aspects of the present example and invention is provided herewith. The figure is intended for illustrative purposes and is not intended to limit the present invention. Depicted in FIG. 7A is one aspect of the invention with triangular valve structures (706) on a first plate (702) and a second plate (704) where the plates are connected by a hinge (700). FIG. 7B depicts another aspect of the invention with square valve structures (708) on a first plate (702) and a second plate (704) where the plates are connected by a hinge (700).

### Example 18

### Apparatus Use in Other Fields Where Fluid Extrusion from a Collapsible Fluid Delivery Bag is Desirable

This aspect of the present invention includes the application of the device to other fields where fluid extrusion from a collapsible fluid delivery bag is desirable. For example, in a scientific laboratory setting, the present invention could be fashioned to the appropriate size such that it applies constant pressure to a reagent contained in a fluid delivery bag. The reagent would be delivered, as desired, under pressure at a significant flow rate and can optionally be controlled by an appropriate structure, such as a valve such as those present on intravenous tubing used in the medical applications described above, Another example would be in the food industry where liquid, or semi-liquid foodstuffs are contained in flexible fluid containers. The present invention would aid extrusion of said foodstuff at an increased rate to a desired location.

### Example 19

### Pin & Cable—Locking Mechanism That Locks Faceplates in the Open Position and Allows Closure When a Trigger Mechanism is Activated

In this example, a Pin & Cable locking mechanism is located within at least one of the faceplates and handles. A small compression spring applies, preferably constant, pressure an upward force on dual pins held within the body of the faceplates, coaxing their advancement up towards the main central hinge. When the faceplates are closed and pressed together, the pins sit within a track but outside of receiver slots. As the faceplates are pulled apart, the pins slide along the track and when the appropriate angle is reached the pins are pushed up into slots at the top of the faceplates. When the pins are recessed into the slots, the faceplates of the device are effectively locked, or jammed, in the open position, preferably approximately 40 degrees apart from one another, but other appropriate angles are appropriate and considered part of the present invention. When activated for closure one pulls the plates apart a small degree (to release the pressure of the main torsion springs on the plates) and presses a trigger button. The trigger button is coupled to a cable which pulls downward on the pins pulling them out of the slots in the faceplate and the large torsion spring takes over, closing the faceplates and extruding the fluid.

FIG. 8A, FIG. 8B and FIG. 8C depict preferred aspects of the present example and invention is provided herewith. The figure is intended for illustrative purposes and is not intended to limit the present invention. Depicted in FIG. 8A is a front view of one of the plates (800). Also depicted is a long handle (802) a trigger button (806) and track for the pin and cable locking mechanism within the plate (804) as is further depicted in FIG. 8A. FIG. 8B depicts a side, cutaway view of FIG. 8A showing internal structures for a pin and cable locking mechanism of FIG. 8C. In FIG. 8B a plate (800) and long handle (802) are shown. Further depicted for the pin and cable structure are the dual pins (804) with a small compression spring (812) with a trigger button (810) that activates a trigger mechanism (814) utilizing a cable (808) that pulls down on the pins. FIG. 8C depicts the pin and cable locking mechanism free of the plate (800) shown in FIG. 8B. Further depicted in FIG. 8C are the dual pins (one of the duel pins is shown (804)), the small compression spring (812), cable (808), trigger mechanism (814), and trigger button (810).

### Example 20

### Rod & Channel Locking Mechanism That Locks Faceplates in the Open Position and Allows Closure When a Trigger Mechanism is Activated

In this example, a Rod & Channel Locking mechanism is located on the sides of the faceplates. The “rod” or crossbar component pivots around a fixed point on one faceplate while the other end slides up and down a guiding channel located on the opposite faceplate. There can be a small torsion spring located behind each crossbar providing a downward tension on the component. When the faceplates are closed and pressed together or substantially together, the free end of the crossbar is held at the top of the channel by the relatively large forces of the main torsion springs. As the faceplates are pulled open to the appropriate degree, depending on the configuration of the device, the free end of the crossbar slides, preferably automatically, down the channel by way of the force provided by the small torsion spring. When the faceplates are pulled open to the appropriate angle, the crossbar operably engages, or finds its way, into a relatively small detent located at the bottom of the channel. This small detent substantially prevents or prevents the crossbar from prematurely sliding back up the channel when one releases the faceplates and effectively locks the plates in the open position. When activated for closure one pulls the plates apart a relatively small degree (to release the pressure of the main torsion springs on the plates), presses a release button trigger mechanism located on the handle of one plate which translates an upwards force onto the crossbar and disengages, or pops it out of, the detent. As the plates are allowed to close together, the force of the relatively large torsion springs take over closing the faceplates and extruding the fluid.

FIG. 9A, FIG. 9B, FIG. 9C, FIG. 9D and FIG. 9F depict preferred aspects of the present example and invention is provided herewith, The figure is intended for illustrative purposes and is not intended to limit the present invention. FIG. 9A is a view of the device from the inside face of the device, whereas FIG. 9B is a view from the outside face of the device, both are shown in the closed position. FIG. 9C is a view of the device from the outside face in the open position, and FIG. 9D is a perspective view from the outside face. FIG. 9E is a close up view of the small notch, or dente (912) of the device. FIG. 9F is a view of an article of manufacture of the present invention including plates.

The figures show the following as depicted and referred to in this example and explained therein. FIG. 9A depicts a crossbar (900), fixed point of crossbar (902), small tension spring (904) that is located at the fixed point of the crossbar, constant tension torsion spring (906), sliding point of crossbar (908), guiding channel or groove (910), small notch or detent (912), trigger (914), a first arm (916) and a second arm (918), and a channel or groove (920) that can provide support for the first arm. FIG. 9B also shows the elements of the sliding point of the crossbar (922) and the fixed point of the crossbar (924) due to the change in view. FIG. 9C, FIG. 9D and FIG. 9E use the same designations as in prior FIG. 9A and FIG. 9B. In FIG. 9E, plates are shown as (926) and (928) and also uses the same designations as in prior FIG. 9A and FIG. 9B.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1. A medical device for administering at least one fluid to at least one subject from at least one flexible fluid container, comprising:
a) at least two plates, each having a front face, a back face, a top edge, a bottom edge, a right edge, and a left edge; wherein the top edges of said at least two plates are operably engaged with said at least one hinge means; and
b) at least one pressure inducing means;
wherein said at least one pressure inducing means is operably engaged with said at least two plates to provide pressure to said at least two plates.

2. The medical device of claim 1, wherein when said at least one flexible fluid container is provided between said at least two plates, pressure is applied to said at least two plates by said at least one pressure inducing means to said at least one flexible fluid container to expel fluid from an outlet of said at least one flexible fluid container.

3. The medical device of claim 1, wherein said at least one hinge means is the same or different and is selected from the group consisting of metal, tape, ties, plastic ties, springs or rings, and can be the same or different or a combination thereof.

4. The medical device of claim 1, wherein said at least one pressure inducing means is selected from the group consisting of one or more clamps, one or more springs, one or more magnets, one or more rubber bands, and one or more bungee cords, and can be the same or different or a combination thereof.

5. The medical device of claim 1, wherein gas from said at least one flexible fluid container does not substantially exit said flexible fluid container.

6. The medical device of claim 1, wherein said at least one hinge means is placed in a position such that medical device does not substantially interfere with at least one hanging apparatus for said at least one fluid delivery bag operably engaged with said medical device.

7. The medical device of claim 1, wherein said at least one hinge means is centrally located.

8. The medical device of claim 1, wherein said at least one hinge means is or are off-centered.

9. The medical device of claim 1, comprising at least one trigger mechanism, the use of which increases the safety and efficiency of said medical device.

10. The medical device of claim 1, comprising at least one trigger mechanism, the use of which increases the safety of said medical device.

11. The medical device of claim 1, comprising at least one trigger mechanism, the use of which increases the efficiency of said medical device.

12. The medical device of claim 1, comprising at least one pin and cable locking mechanism.

13. The medical device of claim 1, comprising at least one rod and channel locking mechanism.

14. A method of administering at least one fluid to at least one subject, comprising:
a) providing at least one subject in need of said at least one fluid;
b) providing at least one medical device of claim 1;
e) providing at least one flexible fluid container comprising said at least one fluid to be administered to said at least one subject; and
d) operably engaging said at least one flexible fluid container with said at least one medical device of claim 1 and said at least one subject to allow the flow of said at least one fluid to said at least one subject from said at least one flexible fluid container.
